(19) United States
(12) Patent Application Publication
(10) Pub. No.: US 2018/0333371 A1
(43) Pub. Date: Nov. 22, 2018

(54) CURCUMIN AND RESVERATROL FOR CHRONIC INFLAMMATION

(71) Applicant: DSM IP Assets B.V., Heerlen (NL)

(72) Inventors: Christopher Michael BUTT, Kaiseraugst (CH); Christoph RIEGGER, Kaiseraugst (CH); Kelly WYNALDA, Kaiseraugst (CH)

(21) Appl. No.: 15/777,700

(22) PCT Filed: Dec. 7, 2016
(86) PCT No.: PCT/EP2016/080014

§ 371 Date: May 21, 2018

Related U.S. Application Data
(60) Provisional application No. 62/266,388, filed on Dec. 11, 2015.

(30) Foreign Application Priority Data
Dec. 15, 2015 (EP) 15200142.6

(51) Int. Cl. A61K 31/12; A61K 31/05; A61P 29/00

(52) U.S. Cl. CPC A61K 31/12; A61P 29/00; A61K 31/05

(57) ABSTRACT

The combination of curcumin and resveratrol can prevent the microglia from participating in the inflammation pathway in the brain. Thus this combination can reduce/prevent inflammation in the brain secondary to conditions such as obesity, diabetes, injury, and infection. The combination can also reduce chronic pain.

### BRIEF DESCRIPTION OF THE INVENTION

This invention relates to the use of the combination of curcumin and resveratrol to decrease chronic inflammation, particularly in the brain where inflammation is largely mediated by microglia cells, and also to reduce chronic pain as perceived by the brain and spinal cord. It has been found that the combination is synergistic in its action on microglia, and thus can be useful in controlling, lessening, and preventing chronic inflammation such as that associated with diabetes, low grade fevers, obesity, hypertension, athletic exertion, and chronic neuropathy.

### BACKGROUND OF THE INVENTION

The combination of curcumin and resveratrol is known to have some synergism in the treatment of certain cancers, such as lung carcinoma. See, e.g., Malhotra et al 2014 PLoS ONE 9(4) e93820, where it was found that the combination regulates p53 phosphorylation at ser 15 and thus enhances the apoptotic that reduce the growth of cancerous cells.

Both curcumin and resveratrol used alone, rather than in combination, are known to have anti-oxidant effects, as discussed in Derochette et al 2013 Chemico-Biological Interactions 206: 186-193. Here, curcumin was found to modulate NADPH oxidase activity better than resveratrol, as curcumin also acts as an oxygen radical scavenger. Resveratrol was found to only have oxygen radical scavenging activity and cannot enter cells as easily as curcumin. Similarly, Cordini et al 2014 Eur. J. Pharmaceutics and Biopharmaceutics 88: 178-185 show that co-encapsulation of both actives improves their antioxidant activities when used as an anti-cancer therapy.

Both curcumin and resveratrol have been proposed as single active ingredients in the treatment of Alzheimer's Disease. See Villaflores et al 2012 Taiwanese J Obstetrics and Gynecology 51:515-525. Additionally, due to its anti-oxidant activity, curcumin has been disclosed to exhibit activity against various other neurological diseases including multiple sclerosis, Parkinson's disease, epilepsy, cerebral injury, age-associated neurodegeneration, schizophrenia, spongiform encephalopathies (Crutzfeldt Jacob aka C-J disease), neuropathic pain and depression. Resveratrol may act as a neuronal protective agent via the SIRT-1 pathway.

Microglia are one class of cells in the brain and spinal cord. They have a function in the central nervous system that is roughly analogous to macrophages in the peripheral immune system. See, e.g. Loane et al 2010 Neurotherapeutics 7(4):366-377. Microglia are able to clear cellular debris and/or toxic substances by phagocytosis, and thus maintain the microenvironment of the CNS. Their cell surfaces have a host of receptors for various growth factors, cytokines, and other factors released by injured cells. In response to this stimulation, the microglia can release pro-inflammatory molecules, including chemokines, which can cause neuronal cell death. Short term activation of microglia is considered beneficial to the well-being of the CNS, however long term activation can be detrimental.

There are numerous situations where chronic inflammation (usually secondary to another condition such as obesity or diabetes) can adversely affect neural tissue. In these situations people with an otherwise healthy CNS (i.e. people without neuronal conditions such as Alzheimer's Disease, dementia, multiple sclerosis, Parkinson's disease, epilepsy, cerebral injury, age-associated neurodegeneration, schizophrenia, or spongiform encephalopathies), but are who experiencing or are at risk of experiencing chronic inflammation in brain tissues would be desirous of protecting these tissues, particularly in situations under which the blood brain barrier is made more permeable.

Microglia are also associated with the production of chronic pain. See, e.g. Giron et al 2015 Pain Management Nursing (Article in press, published on line May 8, 2015) 1-13. Microglia which are activated can release various inflammatory molecules. The painful condition can last long after the injury initiating the pain response has healed. Currently, there is not a suitable treatment that focuses on chronic pain alone which does not have the risk of side effects and/or addiction and it would be desirous to have such a treatment agent.

### DETAILED DESCRIPTION OF THE INVENTION

It has been found, in accordance with this invention that the combination of curcumin and resveratrol can act synergistically as an anti-inflammation agent on microglial cells. Thus, one aspect of this invention is the use of the combination of curcumin and resveratrol to protect brain tissue, particularly microglia cells, from activation, and thus lessen or prevent chronic inflammation. In preferred aspects of this invention, the chronic inflammation experienced is from one of these categories:

a) it is secondary to diabetes or obesity,

b) it is a result of a low level trauma to the brain, such as that experienced from contact sports, fall, or other minor head injury; or

c) it is the result of a low grade fever or infection such as influenza, sinusitis, or meningitis

d) it accompanies hypertension associated inflammation which occurs with blood brain barrier leakage.

Importantly, what is specifically not part of this invention is the use of the combination of curcumin and resveratrol in preventing, curing, and/or lessening the symptoms of any of the following: cancer, Alzheimer's Disease, multiple sclerosis, Parkinson's disease, epilepsy, cerebral injury, age-associated neurodegeneration, schizophrenia, spongiform encephalopathies (C-J disease), neuropathic pain and depression. Further, the use of the combination in persons who have any of the aforementioned diseases to treat chronic inflammation as a result of diabetes, obesity, low level trauma to the brain, low grade fever, or hypertension is also expressly excluded.

Another aspect of this invention is the use of the combination of curcumin and resveratrol to prevent, treat, or lessen the sensation of chronic pain. It is known that over time, opiod pain medications can lose their efficacy. Thus another aspect of this invention is the co-administration of the combination of curcumin and resveratrol to help maintain opoid effectiveness and/or to prevent, treat, or lessen the sensation of chronic pain, with the proviso that the person in need thereof does not also have: cancer, Alzheimer's Disease, multiple sclerosis, Parkinson's disease, epilepsy, cerebral injury, age-associated neurodegeneration, schizophrenia, spongiform encephalopathies (C-J disease), or depression.

Another aspect of this invention is the use of the combination of curcumin and resveratrol to help preserve the normal blood brain barrier (BBB) function as this combination has a synergistic anti-inflammatory effect on microglia cells, which help maintain the integrity of the BBB.

This is particularly important as an adjunct to hypertensive therapies. Thus another aspect of this invention is the use of the combination of curcumin and resveratrol to control inflammation in persons with hypertension.

### BRIEF DESCRIPTION OF THE FIGURES

FIG. 1. Curcumin C3 extract (0-30 nM) inhibited the LPS-stimulated release of inflammatory cytokines by primary microglia. Concentrations of curcumin were calculated based on the manufacturer's documented percentages of the compound in the C3 extract. FIG. 1A: Curcumin C3 treatments >10 nM (18 μg/mL) significantly inhibited PGE₂ release and IL-1β release (FIG. 1B). FIG. 1C: A curcumin C3 concentration of at least 20 nM (50 μg/mL) was required for significant inhibition of IL-6 release. FIG. 1D: Curcumin C3 concentrations >5 nM (12.5 μg/mL) significantly inhibited TNF-α release. Significant differences (p<0.05) between treatments were determined by 1-way ANOVA followed by Tukey's post hoc tests. The ANOVA parameters are inset within each graph. Error bars represent the SEM.

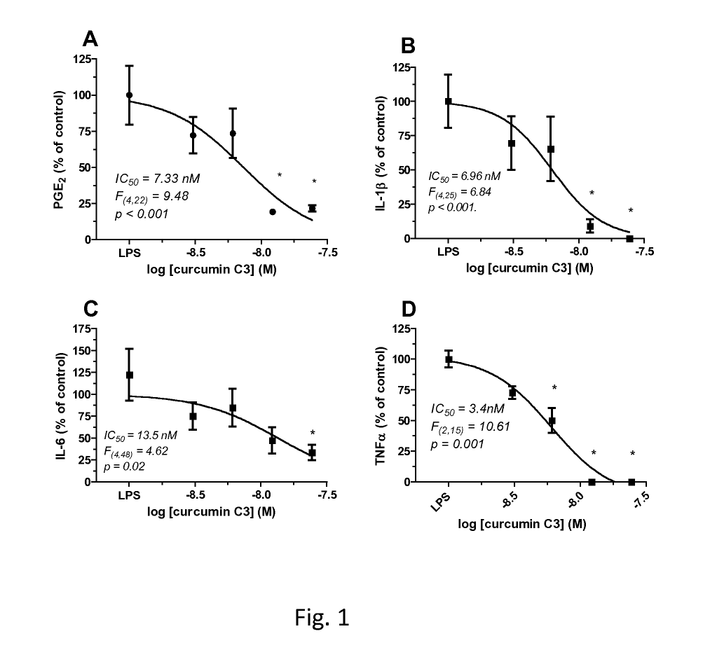

FIG. 2. Resveratrol (0-250 μM) only inhibited the LPS-stimulated expression of PGE₂ by primary microglia. FIG. 2A: Resveratrol significantly inhibited PGE₂ release at 75 μM and 250 μM, but significant effects on TNF-α (FIG. 2B) and IL-1β (FIG. 2C) release were not detected. Significant differences (p<0.05) between treatments were determined by 1-way ANOVA followed by Tukey's post hoc tests. ANOVA parameters are inset within each graph. Error bars represent the SEM.

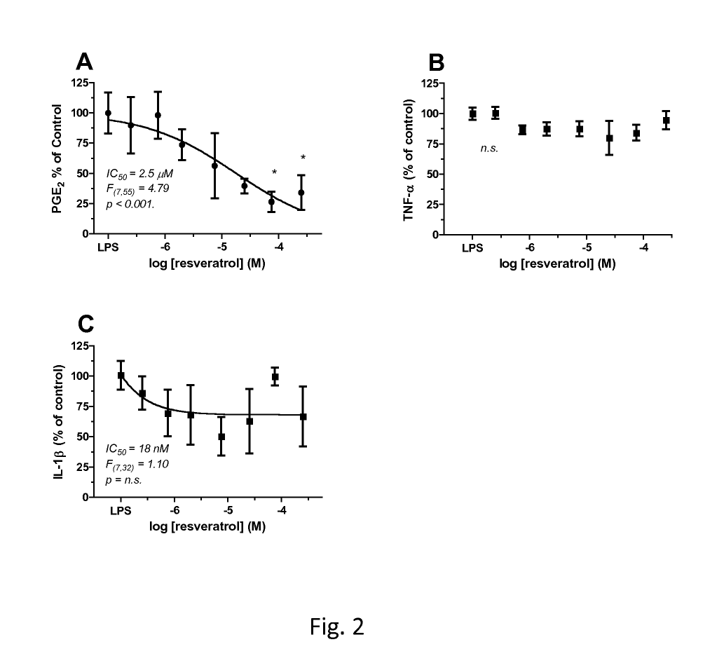

FIG. 3. Combination of resveratrol and curcumin C3 extract at their respective IC₅₀ values inhibited PGE₂ release from primary microglia with likely synergistic effects. Resveratrol (2.25 μM), curcumin C3 (7 nM; 14.4 μg/mL), and combination of resveratrol (2.25 μM)+curcumin C3 (7 nM) inhibited PGE₂ release, with the combination having a significantly stronger effect than the compounds alone. Significant differences (p<0.05) between treatments were determined by 1-way ANOVA followed by Tukey's post hoc tests. Bars that do not have common letters differ significantly. A dotted line at 50% inhibition is shown to highlight the responses of individual compounds in comparison to the combination. ANOVA parameters are inset within the graph. Error bars represent the SEM.

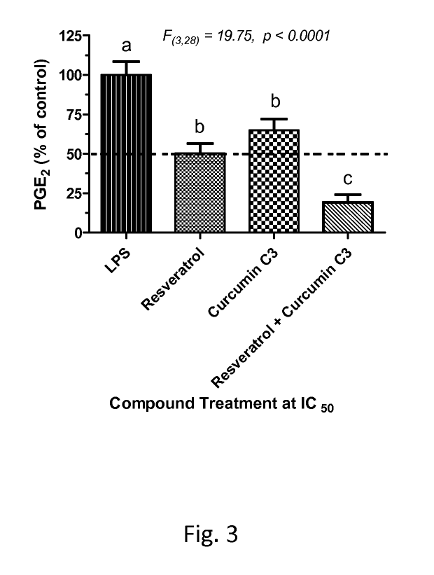

FIG. 4. Synergism analyses of resveratrol (2.25 μM)+curcumin C3 (7 nM) combination detected robust synergism. The affected fraction (Fa, i.e. 90% inhibition=0.9) is plotted versus the combination index values calculated. All calculated values were <0.1, indicative of a very strong synergistic effect of the two compounds, with a mean concentration index value of 0.04. Dose reduction index (DRI) values for resveratrol and curcumin C3 were determined as 42.76 and 21.86, respectively. Therefore, a 43% decrease in resveratrol concentration (i.e. 1.3 μM) and 22% decrease in curcumin C3 concentration (i.e. 6.5 nM) would still have a synergistic effect when put into combination.

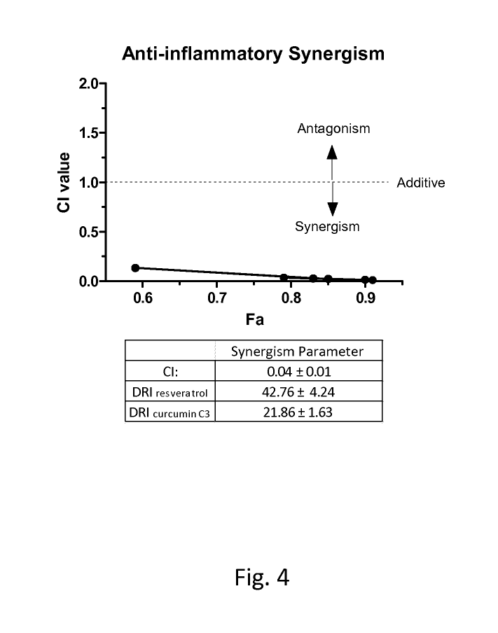

As used throughout the specification and claims the following definitions apply:

“Chronic pain” is pain that lasts more than 12 weeks.

“Acute inflammation” is usually caused by bacterial pathogens or injured tissues. It generally lasts a few days, but may lead to a chronic inflammation.

“Chronic inflammation” is characterized primarily by its persistence and lack of clear resolution. It can be caused by non-degradable pathogens, viral infection, persistent foreign bodies, or auto-immune reactions. It occurs when the tissues are unable to overcome the effects of the injuring agent.

“Healthy individual” as used in this application specifically refers to a person who does not have any of the following diseases/conditions: cancer, Alzheimer's Disease, multiple sclerosis, Parkinson's disease, epilepsy, cerebral injury, age-associated neurodegeneration, schizophrenia, spongiform encephalopathies (C-J disease), and depression.

An “otherwise healthy individual” means the individual has a disease/condition which is to be treated or ameliorated by administration of the combination of this invention, but does not concurrently have any of the following diseases/conditions: cancer, Alzheimer's Disease, multiple sclerosis, Parkinson's disease, epilepsy, cerebral injury, age-associated neurodegeneration, schizophrenia, spongiform encephalopathies (C-J disease), and depression.

Dosages

The dose range for the combination of cucurmin and resveratrol for an adult is from 1 mg to 1000 mg per day Preferably the range of the combination is from 25-500 mg/day, and more preferably the range is from 100-200 mg/day. In some aspects of this invention the combination is administered at 150 mg/day.

The ratio of curcumin:resveratrol should be a ratio which has synergistic effects. This is a rather broad ration and can be as low as 1:138 and as high as 1:6250. For convenience of manufacture, the range should be between 1:100 to 100:1, preferably 50:1 to 1:50, and more preferably from −1:10 to 10:1. A 1:1 ratio can also be envisioned (such as 150-500 mg of each).

The doses are preferably administered once per day, although if desired the dosage can be split into 2 or 3 convenient smaller doses to be administered throughout the day.

For best results, the combination of curcurmin and resveratrol should be taken continuously for at least one week, preferably at least 4 weeks, and may be taken for a longer time.

Forms

The nutraceutical, dietary and/or pharmaceutical compositions according to the present invention may be in any galenic form that is suitable for administering to the human body, especially in any form that is conventional for oral administration, e.g. in solid form, such as (additives/supplements for) food or feed, food or feed premix, fortified food or feed, tablets, pills, granules, dragées, capsules, and effervescent formulations such as powders and tablets, or in liquid form such as solutions, emulsions or suspensions as e.g. beverages, pastes and oily suspensions. The pastes may be encapsulated in hard or soft shell capsules, whereby the capsules feature e.g. a matrix of (fish, swine, poultry, cow) gelatin, plant proteins or ligninsulfonate. Examples for other application forms are forms for transdermal, parenteral or injectable administration. The dietary and pharmaceutical compositions may be in the form of controlled (delayed) release formulations. The dietary compositions according to the present invention may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials.

Examples of food which is suitable for containing the combination of resveratrol and curcumin are cereal bars, dairy products, such as yoghurts, and bakery items, such as cakes and cookies. Examples of fortified food are cereal bars, and bakery items, such as bread, bread rolls, bagels, cakes and cookies. Examples of dietary supplements are tablets, pills, granules, dragées, capsules and effervescent formulations, in the form of non-alcoholic drinks, such as soft drinks, fruit juices, lemonades, near-water drinks, teas and milk-based drinks, in the form of liquid food, such as soups and dairy products (muesli drinks). Also appropriate are beverages. Beverages encompass non-alcoholic and alcoholic drinks as well as liquid preparations to be added to drinking water and liquid food. Non-alcoholic drinks are e.g. soft drinks, sport drinks, fruit juices, vegetable juices (e.g. tomato juice), lemonades, teas and milk-based drinks. Liquid foods are e.g. soups and dairy products (e.g. muesli drinks).

In addition to the combination of resveratrol and curcumin, the pharmaceutical compositions according to the present invention may further contain conventional pharmaceutical additives and adjuvants, excipients or diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like. The carrier material can be organic or inorganic inert carrier material suitable for oral/parenteral/injectable administration.

Diabetes-Related Inflammation

One often-overlooked aspect of diabetes (or a pre-diabetic state, such as hyperglycemia) is chronic inflammation, which can also be present in the brain. By using the combination of cucurmin and resveratrol, the amount of inflammation in the brain can be reduced and/or eliminated. Thus one aspect of this invention is the use of the combination of resveratrol and curcumin to reduce the amount of inflammation in brain and/or other neural tissues which is secondary to diabetes. Another aspect is a method of controlling, reducing, or ameliorating the symptoms of acute or chronic inflammation of the brain and/or other neural tissues which are secondary to diabetes comprising administering the combination of resveratrol and curcumin to a person who has diabetes, with the proviso that the person is an otherwise healthy person.

Another aspect of this invention is the use of the combination of resveratrol and curcumin to prevent, minimize, delay the onset of and/or ameliorate the amount of inflammation in the brain and/or other neural tissues which is secondary to by administration of the combination to a healthy person who is as risk of developing diabetes.

Inflammation Associated with Obesity

Persons who are obese, whether or not they concurrently have diabetes may experience chronic inflammation, including inflammation in the brain and neural tissues. Thus another aspect of this invention is use of the combination of curcumin and resveratrol to prevent, or lessen, or eliminate inflammation in the brain and/or neural tissues which is secondary to obesity, or obesity concurrent with diabetes. Another aspect of this invention is a method of reducing inflammation in the brain or neural tissues which is secondary to obesity comprising administering a combination of resveratrol and curcumin to a person who is obese, with the proviso that the person is an otherwise healthy person.

For persons who are at risk of developing inflammation of the brain and neural tissues, but who do not yet have such inflammation, particularly persons who are overweight, another aspect of this invention is a method of reducing the risk of developing inflammation of the brain and neural tissues secondary to obesity comprising administering the combination of curcumin and resveratrol to otherwise healthy persons.

Inflammation Secondary to Minor Brain Trauma

People who sustain one or more minor head injuries, i.e. one or more minor insults to the head, which is not sufficient to induce a concussion or loss of consciousness, also can experience inflammation of the brain and neural tissues, although there may be no frank signs of injury. Inflammation can occur even when protective headgear is worn. Examples of people at risk for these types of injuries include:
athletes, especially those involved in sports where there is physical contact or the risk of falling or hitting the head. Examples of these sports include: boxing, wrestling, football, rugby, martial arts, skating, skiing, cycling, gymnastics, horseback riding and the like.
people who have jobs which may expose them to minor head injuries such as in the construction industry, stunt performers, military personnel, rescuers, firefighters, police and the like, or
people whose balance is compromised due to another medical condition and are at the risk of falls, including the frail elderly, or those with epilepsy.

Thus another aspect of this invention is the use of the combination of resveratrol and curcumin to prevent, ameliorate or lessen the risk of inflammation of the brain and other neural tissue secondary to minor head trauma in persons as risk of suffering minor head injuries. Another aspect of this invention is a method of reducing the risk of inflammation of the brain and other neural tissues secondary to minor head injuries comprising administering to an otherwise healthy person at risk of sustaining said injuries the combination of curcumin and resveratrol.

Inflammation Secondary to Fevers

People who suffer from low grade fevers often experience inflammation in the brain. While a short term illness of this type may not be particularly serious, prolonged low grade fevers can result in chronic inflammation. For example, people who suffer from prolonged sinusitis or other allergic reactions may experience inflammation in the brain or other neural tissue. Other diseases/conditions which can also impact the brain are those which directly affect the brain and neural tissue itself such as meningitis. Administration to persons suffering from meningitis can reduce the associated inflammation.

Thus another aspect of this invention is a method of reducing the inflammation in the brain which is secondary to a low grade fever or meningitis comprising administering to a person in need thereof a combination of curcumin and resveratrol, with the proviso that the person is not also suffering from cancer, Alzheimer's Disease, multiple sclerosis, Parkinson's disease, epilepsy, cerebral injury, age-associated neurodegeneration, schizophrenia, spongiform encephalopathies (C-J disease), and/or depression. Yet another aspect of this invention is the use of the combination of curcumin and resveratrol to reduce inflammation in the brain which is secondary to a low grade fever or meningitis.

Persons Experiencing Chronic Pain

While chronic pain is itself not technically a disease, it can be debilitating. Further, opoids can offen lose their effectiveness over time. Another aspect of this invention is the use of the combination of resveratrol and curcumin to ameliorate or relieve chronic pain in persons in need thereof and who are not suffering from cancer, Alzheimer's Disease, multiple sclerosis, Parkinson's disease, epilepsy, cerebral injury, age-associated neurodegeneration, schizophrenia, spongiform encephalopathies (C-J disease), and/or depression. Thus this invention also encompasses the method of reducing chronic pain comprising administering a combination of resveratrol and curcumin to person in need thereof, with the provisio that the person does not have concomitant cancer, Alzheimer's Disease, multiple sclerosis, Parkinson's disease, epilepsy, cerebral injury, age-associated neurodegeneration, schizophrenia, spongiform encephalopathies (C-J disease), and/or depression.

Inflammation Secondary to Hypertension

Hypertension can interfere with the integrity of the BBB and thus be involved in the inflammation pathway. Thus another aspect of this invention is the use of the combination of curcumin and resveratrol in persons with hypertension but who are otherwise healthy. Also as part of this invention is the method of reducing inflammation in the brain and neural tissues in a person with hypertension comprising administering a combination of resveratrol and curcumin, with the provision that the person does not have concomitant cancer, Alzheimer's Disease, multiple sclerosis, Parkinson's disease, epilepsy, cerebral injury, age-associated neurodegeneration, schizophrenia, spongiform encephalopathies (C-J disease), and/or depression.

Protection of the Blood-Brain-Barrier

A further aspect of this invention is the use of the combination of curucmin and resveratrol to protect the BBB in persons at risk of BBB leakage, such as persons who are or who are risk of suffering from diabetes, obesity, low level trauma to the brain, a low grade fever or hypertension associated inflammation which occurs with blood brain barrier leakage. Thus another aspect of this invention is a method of protecting the BBB from leakage comprising administering to a person who is or who is at risk of experiencing BBB-leakage a combination of curcumin and resveratrol, with the proviso that the person is otherwise healthy.

The following non-limiting examples are presented to better illustrate the invention.

### EXAMPLES

### Example 1

Materials and Methods

Microglial Harvesting and Culture. All experiments involving animals were approved by the Animal Care and Use Committee established at Bolder Biopath, Inc. (Boulder, Colo.). Primary microglial cultures were derived from neocortical tissue of E22 Sprague Dawley rat pups taken from timed-pregnant dams (Charles River) by Caesarean section. Briefly, the pup brain cortices were isolated in Hibernate EB (Brain Bits; Springfield, Ill.). The samples were then transferred to Hibernate E (without Ca; Brain Bits; Springfield, Ill.) and washed twice with fresh Hibernate E (−Ca). The tissue was next digested in Hibernate E (−Ca) containing 0.5% trypsin (Sigma) for 15 min at 37° C.). The overlying solution was removed, and the trypsin reaction was quenched by washing the settled tissue with Hibernated E (—Ca) containing 20% heat-inactivated fetal bovine serum (FBS; Hyclone).

The tissue was triturated into single cells, and the suspension was filtered through a 70-μm cell strainer. Cell numbers and viability were consistently greater than 95% as determined using trypan blue (0.4%; Fisher Scientific) and a Countess automated cell counter (Life Technologies, Grand Island, N.Y.). Cells were seeded into T-75 flasks (VWR) at 8×10⁶ cells/flask in HiGlutaXL Dulbecco's Modified Eagle Medium (DMEM, 4.5 g/L glucose, plus glutamine; HyClone) containing 100 μg/mL penicillin, 100 μg/mL streptomycin, 0.5 ng/mL amphotericin (CellnTec) and 1 mM sodium pyruvate. The media was changed on day 3 in vitro, and the cells were fed every 4-5 days while in the flasks. At day 14 in vitro, mouse granulocyte macrophage colony stimulating factor (mGM-CSF; R&D Systems; Minneapolis, Minn.) was added at a final concentration of 5 ng/mL to stimulate microglial differentiation.

Cell Stimulation and Cytokine Assays. Microglia were isolated every 2 weeks from the media over the feeder cultures and were seeded into 96-well plates (75,000 cells/well; VWR) in DMEM , 10% FBS and 100 μg/mL penicillin, 100 μg/mL streptomycin, 0.5 ng/mL amphotericin. 24 hours after plating, cells were treated with Curcumin C3 extract (0-50 μg/mL; 0-25 nM; Sabinsa; Langen Hessen, Germany), resveratrol (0-250 μM; ResVida; DSM Nutritional Products) or a combination thereof. An additional 24 hours were then given before cells were stimulated with lipopolysaccharide (LPS; 100 ng/mL; Sigma). 24 hours after stimulation, supernatants were collected for cytokine assays. Supernatants were stored at −20° C. until assays were performed. The concentration of prostaglandin E2 (PGE₂) in the supernatant was measured by ELISA (Cayman Chemical). The concentrations of tumor necrosis factor-α (TNF-α), interleukin-1β (IL-1β) and interleukin-6 (IL-6) in the supernatant were determined by multiplex ELISA (Quansys Biosciences; Logan, Utah). The data were then normalized to a percentage of the control response to LPS alone.

Calculation of Synergism. Synergism arising from the combination of resveratrol with curcumin C3 was determined according to the median-effect principle and classical isobologram equation of Chou-Talay and according to the method of Choi et al (2013).¹ This principle is based on mass-action law and was developed by Talay and Chou² to unify the Michaelis-Menten, Hill, Heanderson-Hasselbach and Schatchard equations, the main theories of biochemistry and biophysics, into a quantitative definition for additive effect, synergism and antagonism when testing drug combinations. Calculations were performed using ComboSyn Software' (ComboSyn, Inc. Paramus, N.J.). Briefly, dose-response curves were plotted for each drug to calculate their respective IC₅₀ values. The IC₅₀ values were then used as the relative Dₘ (dose with 50% effect) values in the synergism calculations. Synergism was confirmed by calculation of the combination index (CI) values. CI values that are close to 1 indicate additive effects, while a CI<1 indicates synergistic effects. When CI>1, antagonistic effects are indicated. Dose reduction index (DRI) values, a measure of the potential fold dose reduction for each of the compounds, when given in combination, compared to the doses of each drug alone are also reported.

1. A method of reducing brain inflammation comprising:
administering to a person experiencing or at risk of experiencing inflammation in the brain or neural tissues resulting from a condition selected from the group consisting of:
Inflammation secondary to diabetes,
Inflammation secondary to obesity,
Inflammation as a result of low level trauma to the brain,
Inflammation as a result of a low grade fever or infection,
Inflammation secondary to hypertension and
Chronic pain
an effective synergistic amount of a combination of resveratrol and curcumin,
with the proviso that the person does not have any of the following diseases: cancer, Alzheimer's Disease, multiple sclerosis, Parkinson's disease, epilepsy, cerebral injury, age-associated neurodegeneration, schizophrenia, spongiform encephalopathies (C-J disease), or depression.

2. A method according to claim 1 wherein the resveratrol and curcumin is administered at a dose of 1-1000 mg per day.

3. The use of a combination of resveratrol and curcumin in combination to reduce or prevent inflammation of the brain and neural system in a person experiencing or at risk of any of the following conditions: Inflammation secondary to diabetes,
Inflammation secondary to obesity,
Inflammation as a result of low level trauma to the brain,
Inflammation as a result of a low grade fever or infection,
Inflammation secondary to hypertension and
Chronic pain.

4. Use according to claim 3 wherein the use is from 1-1000 mg per day, preferably about 25-500 mg/day, and more preferably from 100-200 mg/day.
